(19) United States
(12) Patent Application Publication
(10) Pub. No.: US 2007/0213695 A1
(43) Pub. Date: Sep. 13, 2007

(54) Continuous skin contact handpiece system for cooling during controlled emmission of light and a method thereof

(76) Inventors: Paul Perl, Tel-Aviv (IL); Arriaza Francisco, Sabadell (ES)

Correspondence Address: BROWDY AND NEIMARK, P.L.L.C.;624 NINTH STREET, NW, SUITE 300, WASHINGTON, DC, 20001-5303, US

(21) Appl. No.: 11/370,230

(22) Filed: Mar. 8, 2006

(51) Int. Cl. A61B 18/18; A61N 5/06

(52) U.S. Cl. 606/9; 607/88

(57) ABSTRACT

A skin contact handpiece system for cooling an internal light source and treated skin, during controlled emission of light from the source combines an internal air-cooled light source adapted to controllably emit light towards the treated skin while preserving the absorption characteristic of the emitted light; and a liquid-cooled skin contact adapted to provide a painless dermal treatment and to prevent overheating of the treated skin by the light.

### FIELD OF THE INVENTION

The present invention generally relates to a continuous skin contact cooling handpiece system for cooling during controlled emission of light. The present invention is useful for skin care and treatment.

### BACKGROUND OF THE INVENTION

A flash lamp is an electric glow discharge lamp designed to produce extremely intense, incoherent, full-spectrum white light for very short durations.

The lamp is comprised of a sealed tube, often made of fused quartz, which is filled with a mixture of gases, primarily xenon, and electrodes to carry electrical current to the gas mixture. Additionally, a high voltage power source is necessary to energize the gas mixture; this high voltage is usually stored on a capacitor so as to allow very speedy delivery of very high electrical current when the lamp is triggered.

The electrodes protrude into each end of the tube, and are connected to a capacitor that is charged to a relatively high voltage. This is usually between 100 and 2000 volts, depending on the length of the tube, and the specific gas mixture.

A flash is initiated by first ionizing the gas mixture, then sending a very large pulse of current through the ionized gas. Ionization is necessary to decrease the electrical resistance of the gas so that a pulse measuring as much as thousands of amperes traverse through the tube. The initial ionization pulse, or trigger pulse, may be applied to one of the internal electrodes, or to a metal band or wire that is wrapped around the glass tube. When the trigger pulse is applied, the gas becomes ionized, and the capacitor immediately discharges through the tube. When this current pulse traverses through the tube, it excites electrons surrounding the gas atoms causing them to jump to higher energy levels. The electrons immediately drop back to a lower orbit, producing photons in the process, which results in a “flash” or emission of high energy electromagnetic waves in the range of wave length that preferably goes from ultraviolet to infrared.

The flash that emanates from a flash lamp may be so intense, that it can ignite flammable materials within a short distance of the tube. Carbon nanotubes are particularly susceptible to this spontaneous ignition when exposed to the light from a flashtube. Similar effects may be exploited for use in aesthetic or medical procedures such as hair removal, tattoo removal, epidermis rejuvenation and destroying lesions or moles. Discharge durations for common flashlamps are in the microsecond to a few milliseconds range and can have repetition rates of hundreds of hertz. This discharge of energy is applied to the patient's skin through appropriate devices by an operator or a therapist, as a treatment.

Techniques designed to counter the effects of skin continuous cooling are known in the art.

EP Patent No. 0885629 discloses an apparatus for therapeutic or cosmetic photo-treatment comprises a flash lamp and a lamp operating circuit. The lamp is cooled by water in contact with the lamp which acts as an infra-red filter to reduce skin burning. Light from the lamp reaches the skin through a light guide which has a convex curved end to focus the light and to press away hemoglobin in the target area or has a concave end to reduce pressure on the skin depending on the desired treatment. Relatively long and low power square shaped power pulses drive the lamp to produce light output pulses adapted to the relaxation time of the target structure to maximize the heating of the target whilst minimizing heating of the skin surface. Target structures may be blood vessels or hair follicles.

And WO Patent No. 200405445 relates to a device for treating human skin by means of radiation. The device has housing with a radiation exit opening, a radiation source which is accommodated in the housing, and a radiation path between the radiation source and the radiation exit opening. A radiation filter is provided in the radiation path. According to the invention the radiation filter comprises water which is in solid state at least during an initial phase of operation of the device. The water in solid state has an optical transmission spectrum which corresponds to the optical transmission spectrum of water in liquid state as present in the skin. As a result the water in solid state acts as an ideal filter for the IR light and near IR light, which would otherwise be absorbed by the water in the skin and cause unwanted heating of the skin. An additional advantage of the water in solid state is its relatively high heat absorbing capacity and its ability to cool the skin in case of direct thermal contact with the skin.

In a particular embodiment the device is an epilator for the removal of hairs from the human skin, the radiation source being a flash lamp which generates light pulses having a high energy density and a broad optical spectrum.

A cost-effective continuous skin contact handpiece system adapted for painless treatment, useful for preventing overheating of the skin thus meets a long felt need. Because none of these prior art references disclose a light emission handpiece that utilizes a combination of an air-cooled light source and a liquid-cooled skin contact means, without changing the absorption characteristic of the emitted light. Also, none of the literature cited teaches a liquid-cooled skin contact means comprising a double-layer arrangement of a first and second transparent layer placed at a short distance from each other; such that said second transparent layer is liquid thermoelectrically cooled.

### SUMMARY OF THE INVENTION

It is thus one object of the present invention to provide an efficient continuous skin contact handpiece system for cooling both internal light source and treated skin, during controlled emission of light from said source; wherein said system combines: an internal air-cooled light source adapted to controllably emit light towards said treated skin while preserving the absorption characteristic of said emitted light; and, a liquid-cooled skin contact means adapted to provide a painless dermal treatment and to prevent overheating of said treated skin by said light.

Another aspect of the present invention is a method for continuous skin-contact cooling during controlled emission of light comprising combining: controllably emitting light towards said treated skin while preserving the absorption characteristic of said light by an air-cooled light source; and, applying a liquid-cooled skin contact means to said treated skin, hence providing a painless dermal treatment and preventing overheating of said treated skin.

While the invention is susceptible to various modifications and alternative forms, specific embodiments thereof have been shown by way of example in the drawings and will herein be described in detail. It should be understood, however, that it is not intended to limit the invention to the particular forms disclosed, but on the contrary, the intention is to cover all modifications, equivalents, and alternatives falling within the spirit and scope of the invention as defined by the appended claims.

### BRIEF DESCRIPTION OF THE FIGURES

In order to understand the invention and to see how it may be implemented in practice and by way of non-limiting example only, with reference to the accompanying drawing,

FIG. 1 is a schematic and simplified cross view diagram of a handpiece system according to one embodiment of the present invention; and,

FIG. 2 is a schematic cross view diagram of another preferred embodiment of the device of the present invention.

### DETAILED DESCRIPTION OF PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a continuous skin contact handpiece system for cooling the treated skin during controlled emission of light.

The continuous skin contact cooling allows treatment on any spectrum with a maximum patient comfort.

The term ‘transparent layer’ refers hereinafter to any organic matrix, inorganic matrix or a combination thereof, wherein the organic matter is selected in a non limiting manner form polymers, e.g., polycarbonates, polymethyl metaacrylates (PMMA), and the inorganic matter is selected in a non limiting manner from glass, silica.

The term ‘electrode’ refers hereinafter to any wire or conducting material.

The term ‘about’ refers hereinafter to a tolerance of ±20% of the defined measure.

The ‘thermoelectric cooling’ uses the Peltier effect to create a heat flux between the junction of two different types of materials.

The term ‘while preserving absorption characteristic’ refers hereinafter to the attenuation of the infrared light emission in water that occurs in prior art when the cooling of the light source is performed in water.

The term ‘air’ may use below in a wider scope and hence also refers hereinafter to any cooling fluid, selected in a non-limiting manner from a group comprising air, nitrogen, carbon dioxide, ammonia, or any other cooling fluid, being liquid, gas or a combination thereof, wherein this fluid does not alter significantly the light spectrum.

The term ‘light emission’ refers hereinafter to any electromagnetic radiation of any wavelength, preferably the light emission lies in the range of about 500 to about 800 nm, or in the range of about 600 nm to about 1000 nm, or for infrared light in the range of about 800 nm to about 1800 nm.

The system according to the present invention is adapted for delivering a constant and controlled broadband optical light in the desired light spectrum. The obtained pulsed light increases the efficacy of the treatment by selecting the desired output energy for this given light spectrum.

This system is a single handpiece allowing at least a triple different broadband emission via simple switch manipulation. For example, for visible light, the spectrum emission lies in the range of about 500 to about 800 nm, or in the range of about 600 nm to about 1000 nm, or for infrared light in the range of about 800 nm to about 1800 nm.

The applications of the described system are diverse; with infrared emission for example, skin tightening, stretch marks, collagen stimulation or acne scars can be performed; with visible emission hair removal, pigment and vascular lesions can be treated.

The flash lamp control device includes a circuit that controls lamps operation so that the operator can easily determine the range of wavelengths and the amount of energy that must be emitted by the lamp, not depending on the frequency, rate and the desired time of application.

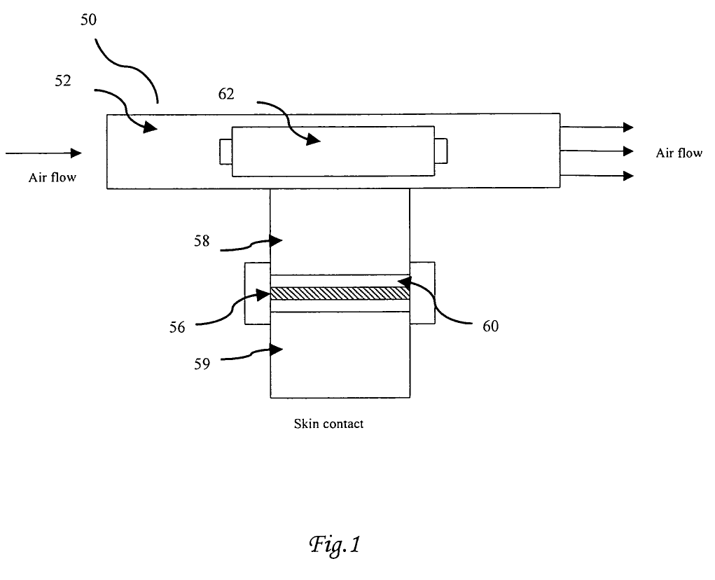

Referring now to the drawings, FIG. 1 is a schematic, simplified and out of scale cross view diagram of the handpiece system. Handpiece 50 preferably includes a housing 52 made of a thermally insulating material such as high temperature plastic, a ceramic material or any other suitable insulator. Said housing forms a cavity filled with a volume of air; a turbine is connected to the housing 52 by a tube for displacing the air into cavity 54. Light source 62 may be any suitable flash lamp or gas discharge arc lamp such as the quartz xenon flash lamp model G5109, commercially available from The Electronic Goldmine, AZ, USA. Filter 56 may be any filter suitable for filtering the radiation which is produced by flash lamp 62 while passing through a portion of a transparent layer 58. Said filter may be a dichroic stained glass filter and can be characterized by a cut-off wavelength of about 495 nm or of about 530 nm. Reflector may be a semi-cylindrical metal-covered device, positioned in the axial direction of the light source. First transparent layer 58 may be positioned adjacent to the light source; whereat second transparent layer 59 is in contact with the surface skin 60.

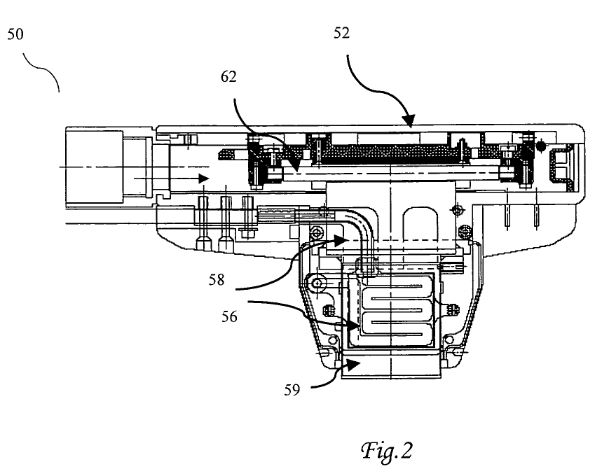

FIG. 2 is a schematic cross view diagram of a preferred embodiment of the device of the present invention. A continuous skin contact handpiece system for cooling during controlled emission of light, is disclosed wherein said system combines an air-cooled light source and a liquid-cooled skin contact means. The novel system is characterized by that the absorption characteristic of the emitted light is not changed; and by that the liquid-cooled skin contact means essentially comprises a double-layer arrangement of a first and a second transparent layer placed at a short distance from each other. The second transparent layer is liquid thermoelectrically cooled. The short distance between the two layers lies in the range of about 0.5 mm to about 6 mm, preferably of about 1 mm to about 3 mm.

The system is useful for preventing overheating of the skin and for painless treatment. This handpiece system comprises hence a light source housing forming a cavity filled with a volume of air; a light source positioned within said housing, providing light for irradiating the skin; a turbine attached to said housing for ventilating said volume of air after is heated by said light source; a reflector positioned in the axial direction of the light source sending light back in the direction of the light source; a trigger circuit characterized by a trigger electrode located adjacent to said light source designed to ionize said light source; applying tension of about 15 kV to the trigger electrode, to activate said light source; applying tension of about 500 V between the electrodes of said light source to ionize the same; said first transparent layer positioned adjacent to said light source; an hermetic chamber confined in the space between said two transparent layers adapted for avoiding penetration of humidity that may be caused by the temperature difference between the two transparent layers; further comprising an optical filter; the second liquid-cooled transparent layer in a thermoelectric manner, being in contact with the treated skin, and, optionally, a computer controlled temperature sensor for adjusting the second transparent layer temperature.

The second thermoelectrically cooled transparent layer is cooled by a Peltier device comprising two plates, a cold one and a hot one. The Peltier hot plate is cooled by means of a cooling liquid that flows in metal conduit. The Peltier cold plate is in contact with said thermoelectrically cooled transparent layer. The two Peltier plates are positioned around said first transparent layer and are pressed against each other by spring mechanism, such that said second transparent layer in contact with the skin is continuously cooled. The hermetic chamber is confined in the space between the two transparent layers by means of a pressure seal, which is mechanically pressed against said two layers. The cold layer and the hot layer are thermally insulated by a gap, eventually filled by air. The seal avoids any humidity in the space between the two layers.

According to another embodiment of the present invention a glass xenon light source can be used. The reflector additionally comprises a heat sinker made of alloys metal, such as brass or any other high thermal conductivity material, and is adapted to increase the light source cooling.

According to another embodiment of the present invention a security mechanism adapted to terminate the light emission in a case that the liquid quantity is not sufficient to cool the second transparent layer is also provided.

According to another embodiment of the present invention, the system also comprises a temperature sensor, especially a Negative Temperature Coefficient (NTC) resistor or any other temperature sensor. The handpiece system is ergonomically designed to be utilized manually. According to a further embodiment of the present invention, the system optionally comprises an additional external transparent layer, in contact with the second transparent layer, which is also continuously cooled. The external transparent layer is adapted to modify properties of the emitted light, especially to modify the broadband spectrum of the emitted light, or to modify the spot size of the emitted light. The spot size can be characterized by any of the following; a length of about 15 mm and a width of about 40 mm, or by a length of about 5 mm and a width of about 10 mm, or by a length of about 10 mm and a width of about 20 mm.

The additional external transparent layer is thermally isolated from the mechanical attachment, but thermally connected to the second layer in order to achieve the cooling of the external transparent layer.

According to another embodiment of the present invention a method for continuous skin-contact cooling during controlled light emission is provided. The method essentially combines an air-cooled light source and a liquid-cooled skin contact means, and characterized by that the absorption characteristic of the emitted light is not changed. The method further provides a liquid-cooled skin contact means, comprising a double-layer arrangement of a first and a second transparent layer placed at a short distance from each other such that the second transparent layer is liquid thermoelectric cooled.

The short distance between the first layer, that heats up due to light transmission from the lamp, and the second layer, cooled by Peltier mechanism, is sealed by the pressure hermetic chamber, where an optical filter attached to the second layer, is inserted.

The method comprises steps of ventilating the volume of air, heated by the light source, in the axial direction of the same; delivering a constant and controlled broadband light for irradiating the treated skin; filtering the light by means of optical filter localized in the hermetic chamber; avoiding penetration of humidity caused by the temperature difference between the two layers placed at a short distance from each other by means of the hermetic chamber confined in-between; cooling the skin-contact transparent layer by thermoelectric cooling; and optionally, adjusting the temperature of the second transparent layer by means of computer controlled temperature sensor.

According to another embodiment of the present invention, the method additionally comprises modifying the spot size of the emitted light, by at least one additional external transparent layer being in contact with the second transparent layer by external attachment mechanism providing thermal insulation between the external transparent layer and mechanical housing. This method is specifically adapted for the treatment of particularly small marks or stains.

According to another embodiment of the present invention, the method additionally comprises thermally isolating the additional external transparent layer from the mechanical attachment, and additionally thermally connecting said additional external transparent layer to the second layer in order to achieve the cooling of the external transparent layer. The cooling temperature lies in the range of about ambient temperature to about minus 10° C.

This method is further adapted for dermal treatments such as pigment lesions or vascular lesions by controllably emitting radiation of an optimized broadband spectrum e.g. about 500 nm to about 800 nm. It is also adapted for hair removal by emitting radiation of an optimized broadband spectrum of about 600 nm to about 1,000 nm and also adapted for skin remodeling or skin tightening by emitting radiation of an optimized broadband spectrum of about 800 nm to about 1,800 nm.

1. A continuous skin contact handpiece system (50) for cooling both internal light source and treated skin, during controlled emission of light from said source; wherein said system combines:
a. an internal air-cooled light source adapted to controllably emit light towards said treated skin while preserving the absorption characteristic of said emitted light; and,
b. a liquid-cooled skin contact means adapted to provide a painless dermal treatment and to prevent overheating of said treated skin by said light.

2. In a continuous skin contact handpiece system (50), a liquid-cooled skin contact means comprises a double-layer arrangement of a first and a second transparent layer placed at a short distance from each other; wherein,
a. said first transparent layer (58) is positioned adjacent to said light source and is heated by the same;
b. a hermetic chamber (60) confined in the space between said two transparent layers is adapted to avoid penetration of humidity that may be caused by the temperature difference between said two transparent layers; and,
c. said second liquid-cooled transparent layer, in contact with said treated skin (59), is cooled in a thermoelectric manner.

3. The system, according to claims 1 or 2; wherein said system combines an air-cooled light source and a liquid-cooled skin contact means, while preserving the absorption characteristic of said emitted light; and further wherein said liquid-cooled skin contact means comprises a double-layer arrangement of a first and a second transparent layer placed at a short distance from each other; such that said second transparent layer is liquid thermoelectrically cooled.

4. The system according to claim 3, comprising:
a. a light source housing (52) forming a cavity filled with a volume of air;
b. a light source, such as a flash lamp (62), positioned within said housing, providing controllable light for irradiating said skin;
c. a turbine attached to said housing for ventilating said volume of air after being heated by said light source;
d. a reflector positioned in the axial direction of the light source sending light back in the direction of the light source;
e. a trigger circuit characterized by a trigger electrode located adjacent to said light source designed to ionize said light source;
f. said first transparent layer (58);
g. said hermetic chamber (60) comprising an optical filter (56);
h. said second liquid-cooled transparent layer (59), and,
i. optionally, a computer controlled temperature sensor for adjusting said second transparent layer temperature.

5. The system according to claim 3, wherein said second liquid-cooled transparent layer is cooled by a Peltier device comprising two plates, a cold plate and a hot plate; said Peltier hot plate is cooled by a cooling liquid that flows in metal conduit; said Peltier cold plate is in contact with said thermoelectrically cooled transparent layer.

6. The system according to claim 3, wherein said two Peltier plates are positioned around said first transparent layer and are pressed against each other by a spring mechanism, such that said second transparent layer in contact with said skin is continuously cooled.

7. The system according to claim 3, wherein said hermetic chamber is confined in the space between said two transparent layers by means of a pressure seal, which is mechanically pressed against said two layers.

8. The system according to claim 3, wherein the light source is a glass xenon flash lamp.

9. The system according to claim 3, wherein said reflector additionally comprises a heat sinker, adapted to increase the light source cooling.

10. The system according to claim 3, additionally comprising a security mechanism adapted to terminate the light emission in the event that the liquid quantity is insufficient to cool said second transparent layer.

11. The system according to claim 3, additionally comprising a temperature sensor, especially an NTC resistor.

12. The system according to claim 3, wherein said handpiece system is ergonomically designed to be utilized manually.

13. The system according to claim 3, comprising at least one additional external transparent layer, in contact with said second transparent layer, which is also continuously cooled; said transparent layer is adapted to modify properties of the emitted light, and especially to modify the broadband spectrum or the spot size of the emitted light.

14. The system according to claim 13, wherein said spot size is characterized by a length of about 5 mm and a width of about 10 mm.

15. The system according to claim 13, wherein said spot size is characterized by a length of about 10 mm and a width of about 20 mm.

16. The system according to claim 13, wherein said additional external transparent layer is thermally isolated from the mechanical attachment, but thermally connected to the second layer in order to achieve the cooling of the external transparent layer.

17. A method for continuous skin-contact cooling during controlled emission of light comprising combining:
a. controllably emitting light towards said treated skin while preserving the absorption characteristic of said light by an air-cooled light source; and,
b. applying a liquid-cooled skin contact means to said treated skin, hence providing a painless dermal treatment and preventing overheating of said treated skin.

18. A method for painless continuous skin-contact cooling while controllably emitting light, comprising
a. providing a liquid-cooled skin contact means; said means is comprised of a double-layer arrangement of a first and a second transparent layer placed at a short distance from each other;
b. positioning said first transparent layer (58) adjacent to said light source;
c. confining a hermetic chamber (60) in the space between said two transparent layers hence avoiding penetration of humidity in between said two layers; and,
d. cooling said second liquid-cooled transparent layer (59) in a thermoelectric manner, while placing it in contact with said treated skin.

19. The method according to claims 17 or 18, comprising:
a. ventilating said volume of air, heated by said light source, in the axial direction of the light source;
b. delivering a constant and controlled broadband light for irradiating said treated skin;
c. filtering said light by an optical filter located in said hermetic chamber;
d. optionally adjusting the temperature of said second transparent layer by means of computer controlled temperature sensor.

20. The method according to claim 19, additionally comprising modifying the spot size of the emitted light, by at least one additional external transparent layer in contact with said second transparent layer by means of an external attachment mechanism providing thermal insulation between said external transparent layer and mechanical housing.

21. The method according to claim 19, additionally comprising thermally isolating said additional external transparent layer from the mechanical attachment, and additionally thermally connecting said additional external transparent layer to the second layer in order to achieve the cooling of the external transparent layer.

22. A method according to claim 19, wherein the cooling temperature lies in the range of room temperature to about minus 10° C.

23. A method according to claim 19, adapted for pigment lesions or vascular lesions, by controllably emitting radiation of an optimized broadband spectrum of about 500 nm to about 800 nm.

24. A method according to claim 19, also adapted for treatments such as hair removal, by emitting radiation of an optimized broadband spectrum of about 600 nm to about 1,000 nm.

25. A method according to claim 19, also adapted for skin remodeling or skin tightening by emitting radiation of an optimized broadband spectrum of about 800 nm to about 1,800 nm.
